(19) United States
(12) Patent Application Publication
(10) Pub. No.: US 2004/0025876 A1
(43) Pub. Date: Feb. 12, 2004

(54) Capsules for dry powder inhalers and methods of making and using same

(76) Inventors: Danforth Miller, Redwood Shores, CA (US); David Lechuga Ballesteros, Santa Clara, CA (US)

Correspondence Address: NEKTAR THERAPEUTICS, 150 INDUSTRIAL ROAD, SAN CARLOS, CA, 94070, US

(21) Appl. No.: 10/430,832

(22) Filed: May 7, 2003

Related U.S. Application Data
(60) Provisional application No. 60/378,703, filed on May 7, 2002.

(51) Int. Cl. A61M 15/00; A61M 16/00

(52) U.S. Cl. 128/203.15; 128/203.12

(57) ABSTRACT

Pulmonary delivery of dry powder formulations by aerosol inhalation has received much attention as an attractive alternative to intravenous, intramuscular, and subcutaneous injection, since this approach eliminates the necessity for injection syringes and needles. The present invention provides dry powder filled cellulose-based capsules having particular utility for use with dry powder inhalers. Such capsules not only readily coordinate with conventional DPIs but also coordinate with conventional powder filling technologies, thereby saving time, labor and cost. The invention further provides a novel procedure for determining, ab initio, appropriate and optimal conditions for preparing such powder filled capsules. Specifically, when packaging dry powder formulations for long-term storage, it is important to ensure that the water content of the powder does not exceed the critical moisture point, that point at which the powder loses physical and chemical stability. The present invention describes means for predicting equilibrium moisture contents, which in turn can be used to establish suitable capsule preparation and filling protocols.

This application claims priority from U.S. Provisional Application Serial No. 60/378,703, filed May 7, 2002 which is incorporated herein by reference.

### FIELD OF THE INVENTION

This invention relates generally to the field of drug delivery, and in particular to the delivery of pharmaceutical formulations to the lungs. More specifically, the invention relates to improvements in unit dose packaging of dry powder formulations, such unit dose packages being in the form of capsules having particular utility for use with dry powder inhalers.

### BACKGROUND OF THE INVENTION

Pulmonary delivery by aerosol inhalation has received much attention as an attractive alternative to intravenous, intramuscular, and subcutaneous injection, since this approach eliminates the necessity for injection syringes and needles. Pulmonary delivery also limits irritation to the skin and body mucosa which are common side effects of transdermally, iontophoretically, and intranasally delivered drugs, eliminates the need for nasal and skin penetration enhancers (typical components of intranasal and transdermal systems that often cause skin irritation/dermatitis), is economically attractive, is amenable to patient self-administration, and is often preferred by patients over other alternative modes of administration.

Of particular interest to the present invention are pulmonary delivery devices which rely on the inhalation of a pharmaceutical formulation by the patient so that the active drug within the dispersion can reach the distal (alveolar) regions of the lung. A variety of aerosolization systems have been proposed to disperse pharmaceutical formulations. Examples of aerosolization systems include DPIs (dry powder inhalers), MDIs (metered dose inhalers, typically including a drug that is stored in a propellant), nebulizers (which aerosolize liquids using compressed gas, usually air), and the like.

The present invention more particularly relates to “dry powder inhalers” or DPIs. DPIs come in two forms: those that utilize an active force, such as a pressurized gas or vibrating or rotating elements, to disperse and aerosolize a drug formulation contained within the device (i.e., active dry powder inhalers) and those that rely exclusively upon the patient's inspiratory effort to disperse and aerosolize a drug formulation contained within the device (i.e., passive dry powder inhalers).

Examples of active powder dispersion devices are described in U.S. Pat. Nos. 5,785,049 and 5,740,794, the disclosures of which are herein incorporated by reference. Additional examples of active DPIs known in the art are disclosed, for example, in U.S. Pat. Nos. 5,875,776, 6,116,238, and 6,237,591 herein incorporated in their entirety by reference, and in co-pending U.S. application Ser. Nos. 09/004,558 filed Jan. 8, 1998; 09/312,434 filed Jun. 4, 1999; 60/136,518 filed May 28, 1999; and 60/141,793 filed Jun. 30, 1999, all of which are hereby incorporated in their entirety by reference.

With regard to passive dry powder inhalers, the inspired gases disperse the pharmaceutical formulation. In this way, the patient's own inhalation is able to provide the energy needed to aerosolize the formulation. This ensures that aerosol generation and inhalation are properly synchronized. However, utilization of the patient's inspiratory effort can be challenging in several respects. For example, for some pharmaceutical formulations, such as those that contain insulin, it may be desirable to limit the inhalation flow rate within certain limits. For example, PCT/US99/04654, filed Mar. 11, 1999, provides for the pulmonary delivery of insulin at gas flow rates less than 17 liters per minute. As another example, co-pending U.S. patent application Ser. No. 09/414,384 describes pulmonary delivery techniques where a high flow resistance is provided for an initial period followed by a period of lower flow resistance. The complete disclosures of all the above references are herein incorporated by reference. Problems associated with variability among patient inspiratory efforts have been addressed through design modifications of dry powder inhaler devices. For example, WO 01/00263 and WO 00/21594, hereby incorporated in their entirety by reference, disclose dry powder inhalers including flow regulation and flow resistance modulation. Other suitable passive DPIs are disclosed in U.S. Pat. Nos. 4,995,385 and 5,727,546, hereby incorporated in their entirety by reference.

For dry powder inhalers to properly function, the chemical and physical characteristics of the respirable dry powder to be dispensed must be carefully designed and maintained. For example, the active agent within a respirable dry powder must be formulated so that it readily disperses into discrete particles. The particles preferably have a mass median diameter (MMD) between 0.5 to 20 μm, preferably 0.5 to 5 μm, and an aerosol particle size distribution whose mass median aerodynamic diameter (MMAD) is less than about 10 μm, more preferably less than 5.0 μm. The mass median aerodynamic diameters of the powders will characteristically range from about 0.5-10 μm MMAD, preferably from about 0.5-5.0 μm MMAD, more preferably from about 1.0-4.0 μm MMAD.

Likewise, the particles need to have a very low bulk density, wherein the minimum powder mass that can be filled into a unit dose container is reduced, which eliminates the need for carrier particles. That is, the relatively low density of the powders of the present invention provides for the reproducible administration of relatively low dose pharmaceutical compounds. Moreover, the elimination of carrier particles will potentially minimize throat deposition and any “gag” effect, since the large carrier particles, typically lactose, will impact the throat and upper airways due to their size.

Accordingly, physical instability such as crystallization or particle agglomeration can substantially undermine operability. To prevent such breakdown of the powders, DPI formulations are typically packaged in single dose units, such as blister packs, foils and the like disclosed in the above mentioned patents. The primary function of the packaging is to extend the shelf life of the respirable dry powders by maintaining the initial powder parameters, to the extent possible, while under standard storage conditions.

Unfortunately, foil and other blister pack dosage forms presently utilized often do not coordinate with the dry powder dispenser. In fact, most commercially available dry powder dispensers are designed for use with puncturable capsules and the like. Accordingly, complex and costly modifications are required to facilitate the use of such blister packs with conventional dry powder dispensers. Thus, capsules are considered desirable due to their compatibility with available inhalation devices and the ability to deliver larger volumes of powder.

### SUMMARY OF THE INVENTION

The present inventors have discovered that by formulating powders for use in capsules, the moisture content of the powder can be controlled by utilizing the capsule as a moisture buffer. By formulating the dry powder for use with a capsule rather than a blister or foil pack, one can utilize conventional technologies in powder filling and dispensing, thereby saving time, labor and cost. Moreover, the capsule preparation method described herein ensures both capsule reliability and formulation stability throughout the shelf life of the packaged product. As shown herein, the present formulation strategy results in improvements in storage stability, namely in the reduction of moisture transfer to the powders, a process that ultimately results in instability and inoperability of the powders. More particularly, the present inventors have discovered that pre-equilibrating the capsule at a pre-determined relative humidity prior to filling minimizes the change in the water content of the powder and ensures that the powder is maintained between its minimum and maximum critical moisture points over an extended period of time.

The present invention is directed to capsules containing dispersible dry powder compositions and methods for using the same. The invention is based, at least in part, on the discovery of the benefits of capsule materials, as compared to traditional foil or blister packaging, in terms of coordination with existing technology and maintenance of storage stability. One of these benefits is the ability of the capsule to maintain the powder within a range of suitable moisture content (i.e., below a maximum critical moisture point and above a minimum critical moisture point) over an extended period of time without the need for an additional desiccant or the like. The use of a capsule to control the water content by acting as a moisture “sink” leads to significant improvements in the dispersibility and flowability of dry powders, which, in turn, leads to the potential for highly efficient delivery of the active agent contained within the formulation, for example to the deep lung and increased in-lung pulmonary bioavailability.

The present invention is further directed to a novel procedure for determining, ab initio, the appropriate and optimal capsule preparation and filling conditions. Specifically, the method of the present invention enables the prediction of optimum RH conditions under which capsules should be prepared and filled, to thereby ensure that the final moisture content of a powder, after it has come to moisture transfer equilibrium with its capsule, is within a range of the critical moisture points of the powder (i.e., below the point at which a powder's physical and chemical stability is compromised and above the point at which the powder's dispersibilty is compromised).

Accordingly, it is an object of the invention to provide a unit dose package comprising (a) a dry powder formulation having a maximum critical moisture point and (b) a capsule receiving said dry powder formulation therein and having an initial moisture content pre-selected such that the equilibrium moisture content of the powder does not exceed the maximum critical moisture point, wherein the formulation is storage stable within said capsule at room temperature.

Accordingly, it is another object of the invention to provide a unit dose package comprising (a) a dry powder formulation having a minimum critical moisture point and (b) a capsule receiving said dry powder formulation therein and having an initial moisture content pre-selected such that the equilibrium moisture content of the powder does not fall below the minimum critical moisture point, wherein the formulation is storage stable within said capsule at room temperature.

It is a further object of the present invention to provide a method of preparing a capsule with a dry powder formulation comprising the steps of:

(1) pre-equilibrating the capsule below a maximum relative humidity (RH), wherein the maximum relative humidity is pre-determined from the masses and moisture sorption isotherms of the powder formulation and the capsule; and

(2) filling the capsule with the dry powder formulation at a relative humidity preselected such that the equilibrium moisture content of the powder does not exceed its maximum critical moisture point, thereby ensuring the storage stability of the powder filled capsule at room temperature.

In one embodiment, the pre-determined maximum relative humidity is less than 50% RH at 25° C. In other embodiments, the pre-determined maximum relative humidity is less than 30% or 20% RH at 25° C.

In one embodiment, the maximum critical moisture content of the powder is less than 4 wt % water. In an alternate embodiment, the maximum critical moisture content of the powder is less than 3 wt % water.

It is a further object of the present invention to provide a method of preparing a capsule with a dry powder formulation comprising the steps of:

(1) pre-equilibrating the capsule above a minimum relative humidity (RH), wherein the minimum relative humidity is pre-determined from the masses and moisture sorption isotherms of the powder formulation and the capsule; and

(2) filling the capsule with the dry powder formulation at a relative humidity preselected such that the equilibrium moisture content of the powder does not fall below its minimum critical moisture point, thereby ensuring the dispersibility of the powder from the capsule after storage at room temperature.

In one embodiment, the pre-determined minimum relative humidity is above 5% RH at 25° C. In another embodiment, the pre-determined minimum relative humidity is above 10% RH at 25° C.

It is a further object to provide capsules containing amorphous, respirable, dispersible dry powder compositions and methods for pulmonary administration to the respiratory tract for local or systemic therapy via aerosolization. It is a further object of the present invention to provide a dry powder inhaler assembly comprising: the unit dose package described above, an actuable perforating element to enable access to the contents of the capsule to release the dry powder formulation contained therein, an inhalation chamber for receiving the dry powder formulation contained within the capsule upon actuation of the perforating element, and a mouthpiece in fluid communication with the inhalation chamber through which the released dry powder formulation is inspired into a patient's lungs, wherein the formulation cannot be dispensed through the mouthpiece until the perforating element is actuated.

In a preferred embodiment, the perforating element is hand-actuated. For example, the perforating element may be actuated by a rotational twisting motion, by a horizontal sliding motion or by the interconnection of mating screw threads. Such perforating elements are known in the inhaler patents cited above. These and other objects and features of the invention will become more fully apparent when the following detailed description is read in conjunction with the accompanying figures and examples.

### DRAWINGS

FIG. 1 depicts a schematic representation of the capsule and powder under initial conditions and upon establishment of equilibrium.

FIGS. 2A and 2B depict the drying rate and hydration rate, respectively, for assembled empty HPMC capsules.

FIG. 3 depicts the moisture sorption isotherms for three samples of Ciprofloxacin/Pulmosphere® powders.

FIGS. 4, 5, and 6 depict the DVS time course for sorption for Ciprofloxacin samples A, B, and C, respectively.

FIG. 7 depicts the SDMT model predictions of the equilibrium content for each Ciprofloxacin powder (Samples A, B, and C) after filling into HPMC capsules that have been pre-equilibrated at various RH values. The average initial RH of these powders is about 15%. This corresponds to about 1.5 to 2.0 wt % water.

FIG. 8 depicts the SDMT model predictions of the equilibrium water content of Ciprofloxacin Sample A, after filling into HPMC capsules that have been pre-equilibrated at various RH values.

FIG. 9 depicts the effect of initial water content of Ciprofloxacin Sample A on its post-filling equilibrium water content.

FIG. 10 depicts the predicted equilibrium water content of the powder after filling into HPMC capsules that have been pre-equilibrated at various RH values. For typical powder masses (1 to 20 mg), the fill mass has only modest effect on the equilibrium water content of the powder.

FIG. 11 compares the measured and predicted changes in water content of the powder and capsule after filling.

### DEFINITIONS

The term “respirable dry powder” refers to a composition that contains finely dispersed particles that are relatively free flowing and capable of (i) being readily dispersed in an inhalation device and (iii) inhaled by a subject so that a portion of the particles reaches the lungs to permit penetration to the alveoli. The dry powder may be crystalline, amorphous or a mixture of both (partially crystalline). Such a powder is considered to be “respirable” or “inhalable”, more particularly, suitable for pulmonary delivery. A dry powder typically contains less than about 20 wt % water, preferably less than 15 wt % water, and more preferably contains less than about 8 wt % water. Although a preferred embodiment is directed to respirable dry powder formulations, it is to be understood that the present invention may be practiced for formulations intended for other routes of administration, such as oral administration.

As used herein, “passive dry powder inhaler” refers to an inhalation device which relies upon the patient's inspiratory effort to disperse and aerosolize a drug formulation contained within the device and does not include inhaler devices which comprise a means for providing energy to disperse and aerosolize the drug formulation, such as pressurized gas and vibrating or rotating elements.

Conversely, an “active dry powder inhaler” refers to an inhalation device which utilizes an active force, such as a compressed gas or the like, to disperse and aerosolize a drug formulation contained within the device.

As used herein, the term “emitted dose” or “ED” refers to an indication of the delivery of a drug formulation from a suitable inhaler device after a firing or dispersion event. More specifically, for dry powder formulations, the ED is a measure of the percentage of powder which is drawn out of a unit dose package and which exits the mouthpiece of an inhaler device. The ED is defined as the ratio of the dose delivered by an inhaler device to the nominal dose (i.e., the mass of powder per unit dose placed into a suitable inhaler device prior to firing). The ED is an experimentally-measured parameter, and is typically determined using an in-vitro device set up which mimics patient dosing. To determine an ED value, a nominal dose of dry powder, typically in unit dose form, is placed into a suitable dry powder inhaler (such as that described in U.S. Pat. No. 4,995,385) which is then actuated, dispersing the powder. The resulting aerosol is then drawn by vacuum from the device, where it is captured on a tared filter attached to the device mouthpiece. The amount of powder that reaches the filter constitutes the emitted dose. For example, for a 5 mg, dry powder-containing dosage form placed into an inhalation device, if dispersion of the powder results in the recovery of 4 mg of powder on a tared filter as described above, then the emitted dose for the dry powder composition is: 4 mg (delivered dose)/5 mg (nominal dose)×100%=80%. For non-homogenous powders, ED values provide an indication of the delivery of drug from an inhaler device after firing rather than of dry powder, and are based on amount of drug rather than on total powder weight. Similarly for MDI and nebulizer dosage forms, the ED corresponds to the percentage of drug which is drawn from a unit dosage form and which exits the mouthpiece of an inhaler device.

As used herein, the term “aerosolized” refers to a gaseous suspension of fine dry powder or liquid particles. An aerosolized medicament may be generated by a dry powder inhaler, a metered dose inhaler, or a nebulizer.

A “dispersible” powder is one having an ED value of at least about 30%, preferably at least about 40%, more preferably at least about 50%, and even more preferably at least about 55%.

“Active agent” as described herein includes an agent, drug, compound, composition of matter or mixture thereof which provides some diagnostic, prophylactic, or pharmacologic, often beneficial, effect. This includes foods, food supplements, nutrients, drugs, vaccines, vitamins, and other beneficial agents. As used herein, the terms further include any physiologically or pharmacologically active substance that produces a localized or systemic effect in a patient. Examples of pharmaceutically active agents include β₂-agonists, steroids such as glucocorticosteroids (preferably anti-inflammatories), anti-cholinergics, leukotriene antagonists, leukotriene synthesis inhibitors, pain relief drugs generally such as analgesics and anti-inflammatories (including both steroidal and non-steroidal anti-inflammatories), cardiovascular agents such as cardiac glycosides, respiratory drugs, anti-asthma agents, bronchodilators, anti-cancer agents, alkaloids (eg, ergot alkaloids) or triptans such as sumatriptan or rizatriptan that can be used in the treatment of migraine, drugs (for instance sulphonyl ureas) useful in the treatment of diabetes and related disorders, sleep inducing drugs including sedatives and hypnotics, psychic energizers, appetite suppressants, anti-arthritics, anti-malarials, anti-epileptics, anti-thrombotics, anti-hypertensives, anti-arrhythmics, anti-oxicants, antidepressants, anti-psychotics, anxiolytics, anti-convulsants, anti-emetics, anti-infectives, anti-histamines, anti-fungal and anti-viral agents, drugs for the treatment of neurological disorders such as Parkinson's disease (dopamine antagonists), drugs for the treatment of alcoholism and other forms of addiction, drugs such as vasodilators for use in the treatment of erectile dysfunction, muscle relaxants, muscle contractants, opioids, stimulants, tranquilizers, antibiotics such as macrolides, aminoglycosides, fluoroquinolones and beta-lactams, vaccines, cytokines, growth factors, hormonal agents including contraceptives, sympathomimetics, diuretics, lipid regulating agents, antiandrogenic agents, antiparasitics, anticoagulants, neoplastics, antineoplastics, hypoglycemics, nutritional agents and supplements, growth supplements, antienteritis agents, vaccines, antibodies, diagnostic agents, and contrasting agents and mixtures of the above (for example the asthma combination treatment containing both steroid and β-agonist).

More particularly, the active agent may fall into one of a number of structural classes, including but not limited to small molecules (preferably insoluble small molecules), peptides, polypeptides, proteins, polysaccharides, steroids, nucleotides, oligonucleotides, polynucleotides, fats, electrolytes, and the like.

Specific examples include the β₂-agonists salbutamol (eg, salbutamol sulphate) and salmeterol (eg, salmeterol xinafoate), the steroids budesonide and fluticasone (eg, fluticasone propionate), the cardiac glycoside digoxin, the alkaloid anti-migraine drug dihydroergotamine mesylate and other alkaloid ergotamines, the alkaloid bromocriptine used in the treatment of Parkinson's disease, sumatriptan, rizatriptan, naratriptan, frovatriptan, almotriptan, zolmatriptan, morphine and the morphine analogue fentanyl (eg, fentanyl citrate), glibenclamide (a sulphonyl urea), benzodiazepines such as vallium, triazolam, alprazolam, midazolam and clonazepam (typically used as hypnotics, for example to treat insomnia or panic attacks), the anti-psychotic agent risperidone, apomorphine for use in the treatment of erectile dysfunction, the anti-infective amphotericin B, the antibiotics tobramycin, ciprofloxacin and moxifloxacin, nicotine, testosterone, the anti-cholenergic bronchodilator ipratropium bromide, the bronchodilator formoterol, monoclonal antibodies and the proteins LHRH, insulin, human growth hormone, calcitonin, interferon (eg, β- or γ-interferon), EPO and Factor VIII, as well as in each case pharmaceutically acceptable salts, esters, analogues and derivatives (for instance prodrug forms) thereof.

Additional examples of active agents suitable for practice with the present invention include but are not limited to aspariginase, amdoxovir (DAPD), antide, becaplermin, calcitonins, cyanovirin, denileukin diftitox, erythropoietin (EPO), EPO agonists (e.g., peptides from about 10-40 amino acids in length and comprising a particular core sequence as described in WO 96/40749), dornase alpha, erythropoiesis stimulating protein (NESP), coagulation factors such as Factor VIIa, Factor VIII, Factor IX, von Willebrand factor; ceredase, cerezyme, alpha-glucosidase, collagen, cyclosporin, alpha defensins, beta defensins, exedin-4, granulocyte colony stimulating factor (GCSF), thrombopoietin (TPO), alpha-1 proteinase inhibitor, elcatonin, granulocyte macrophage colony stimulating factor (GMCSF), fibrinogen, filgrastim, growth hormones, growth hormone releasing hormone (GHRH), GRO-beta, GRO-beta antibody, bone morphogenic proteins such as bone morphogenic protein-2, bone morphogenic protein-6, OP-1; acidic fibroblast growth factor, basic fibroblast growth factor, CD-40 ligand, heparin, human serum albumin, low molecular weight heparin (LMWH), interferons such as interferon alpha, interferon beta, interferon gamma, interferon omega, interferon tau; interleukins and interleukin receptors such as interleukin-1 receptor, interleukin-2, interluekin-2 fusion proteins, interleukin-1 receptor antagonist, interleukin-3, interleukin-4, interleukin-4 receptor, interleukin-6, interleukin-8, interleukin-12, interleukin-13 receptor, interleukin-17 receptor; lactoferrin and lactoferrin fragments, luteinizing hormone releasing hormone (LHRH), insulin, pro-insulin, insulin analogues (e.g., mono-acylated insulin as described in U.S. Pat. No. 5,922,675), amylin, C-peptide, somatostatin, somatostatin analogs including octreotide, vasopressin, follicle stimulating hormone (FSH), influenza vaccine, insulin-like growth factor (IGF), insulintropin, macrophage colony stimulating factor (MCSF), plasminogen activators such as alteplase, urokinase, reteplase, streptokinase, pamiteplase, lanoteplase, and teneteplase; nerve growth factor (NGF), osteoprotegerin, platelet-derived growth factor, tissue growth factors, transforming growth factor-1, vascular endothelial growth factor, leukemia inhibiting factor, keratinocyte growth factor (KGF), glial growth factor (GGF), T Cell receptors, CD molecules/antigens, tumor necrosis factor (TNF), monocyte chemoattractant protein-1, endothelial growth factors, parathyroid hormone (PTH), glucagon-like peptide, somatotropin, thymosin alpha 1, thymosin alpha 1 IIb/IIIa inhibitor, thymosin beta 10, thymosin beta 9, thymosin beta 4, alpha-1 antitrypsin, phosphodiesterase (PDE) compounds, VLA-4 (very late antigen-4), VLA-4 inhibitors, bisphosponates, respiratory syncytial virus antibody, cystic fibrosis transmembrane regulator (CFTR) gene, deoxyreibonuclease (Dnase), bactericidal/permeability increasing protein (BPI), and anti-CMV antibody. Exemplary monoclonal antibodies include etanercept (a dimeric fusion protein consisting of the extracellular ligand-binding portion of the human 75 kD TNF receptor linked to the Fc portion of IgGI), abciximab, afeliomomab, basiliximab, daclizumab, infliximab, ibritumomab tiuexetan, mitumomab, muromonab-CD3, iodine 131 tositumomab conjugate, olizumab, rituximab, and trastuzumab (herceptin), amifostine, amiodarone, aminoglutethimide, amsacrine, anagrelide, anastrozole, asparaginase, anthracyclines, bexarotene, bicalutamide, bleomycin, buserelin, busulfan, cabergoline, capecitabine, carboplatin, carmustine, chlorambucin, cisplatin, cladribine, clodronate, cyclophosphamide, cyproterone, cytarabine, camptothecins, 13-cis retinoic acid, all trans retinoic acid; dacarbazine, dactinomycin, daunorubicin, dexamethasone, diclofenac, diethylstilbestrol, docetaxel, doxorubicin, epirubicin, estramustine, etoposide, exemestane, fexofenadine, fludarabine, fludrocortisone, fluorouracil, fluoxymesterone, flutamide, gemcitabine, epinephrine, L-Dopa, hydroxyurea, idarubicin, ifosfamide, imatinib, irinotecan, itraconazole, goserelin, letrozole, leucovorin, levamisole, lomustine, mechlorethamine, medroxyprogesterone, megestrol, melphalan, mercaptopurine, methotrexate, metoclopramide, mitomycin, mitotane, mitoxantrone, naloxone, nicotine, nilutamide, octreotide, oxaliplatin, pamidronate, pentostatin, pilcamycin, porfimer, prednisone, procarbazine, prochlorperazine, ondansetron, raltitrexed, sirolimus, streptozocin, tacrolimus, tamoxifen, temozolomide, teniposide, testosterone, tetrahydrocannabinol, thalidomide, thioguanine, thiotepa, topotecan, tretinoin, valrubicin, vinblastine, vincristine, vindesine, vinorelbine, dolasetron, granisetron; formoterol, fluticasone, leuprolide, midazolam, alprazolam, amphotericin B, podophylotoxins, nucleoside antivirals, aroyl hydrazones, sumatriptan; macrolides such as erythromycin, oleandomycin, troleandomycin, roxithromycin, clarithromycin, davercin, azithromycin, flurithromycin, dirithromycin, josamycin, spiromycin, midecamycin, leucomycin, miocamycin, rokitamycin, andazithromycin, and swinolide A; fluoroquinolones such as ciprofloxacin, ofloxacin, levofloxacin, trovafloxacin, alatrofloxacin, moxifloxicin, norfloxacin, enoxacin, grepafloxacin, gatifloxacin, lomefloxacin, sparfloxacin, temafloxacin, pefloxacin, amifloxacin, fleroxacin, tosufloxacin, prulifloxacin, irloxacin, pazufloxacin, clinafloxacin, and sitafloxacin; aminoglycosides such as gentamicin, netilmicin, paramecin, tobramycin, amikacin, kanamycin, neomycin, and streptomycin, vancomycin, teicoplanin, rampolanin, mideplanin, colistin, daptomycin, gramicidin, colistimethate; polymixins such as polymixin B, capreomycin, bacitracin, penems; penicillins including penicilinase-sensitive agents like penicillin G, penicillin V; penicilinase-resistant agents like methicillin, oxacillin, cloxacillin, dicloxacillin, floxacillin, nafcillin; gram negative microorganism active agents like ampicillin, amoxicillin, and hetacillin, cillin, and galampicillin; antipseudomonal penicillins like carbenicillin, ticarcillin, azlocillin, mezlocillin, and piperacillin; cephalosporins like cefpodoxime, cefprozil, ceftbuten, ceftizoxime, ceftriaxone, cephalothin, cephapirin, cephalexin, cephradrine, cefoxitin, cefamandole, cefazolin, cephaloridine, cefaclor, cefadroxil, cephaloglycin, cefuroxime, ceforanide, cefotaxime, cefatrizine, cephacetrile, cefepime, cefixime, cefonicid, cefoperazone, cefotetan, cefmetazole, ceftazidime, loracarbef, and moxalactam, monobactams like aztreonam; and carbapenems such as imipenem, meropenem, pentamidine isethiouate, albuterol sulfate, lidocaine, metaproterenol sulfate, beclomethasone diprepionate, triamcinolone acetamide, budesonide acetonide, fluticasone, ipratropium bromide, flunisolide, cromolyn sodium, and ergotamine tartrate; taxanes such as paclitaxel; SN-38, and tyrphostines.

The above exemplary biologically active agents are meant to encompass, where applicable, analogues, agonists, antagonists, inhibitors, isomers, and pharmaceutically acceptable salt forms thereof. In reference to peptides and proteins, the invention is intended to encompass synthetic, recombinant, native, glycosylated, non-glycosylated, and biologically active fragments and analogs thereof. Active agents may further comprise nucleic acids, present as bare nucleic acid molecules, viral vectors, associated viral particles, nucleic acids associated or incorporated within lipids or a lipid-containing material, plasmid DNA or RNA or other nucleic acid construction of a type suitable for transfection or transformation of cells, particularly cells of the alveolar regions of the lungs. The active agents may be in various forms, such as free base, soluble and insoluble charged or uncharged molecules, components of molecular complexes or pharmacologically acceptable salts. The active agents may be naturally occurring molecules or they may be recombinantly produced, or they may be analogs of the naturally occurring or recombinantly produced active agents with one or more amino acids added or deleted. Further, the active agent may comprise live attenuated or killed viruses suitable for use as vaccines.

A “dispersing agent” refers to a component of the respirable dry powder formulation described herein that is effective, when present, from 0.01 to 99 percent by weight of the composition, preferably from 0.01 to 70 percent by weight, to increase the dispersibility of the respirable dry powder formulation (determined by emitted dose determination) by at least 10% when compared to the dispersibility of the respirable dry powder formulation absent the dispersing agent. Suitable dispersing agents are disclosed in PCT applications WO 95/31479, WO 96/32096, and WO 96/32149, hereby incorporated in their entirety by reference. As described therein, suitable agents include water-soluble polypeptides and hydrophobic amino acids such as tryptophan, leucine, phenylalanine, and glycine. Leucine is particularly preferred for use according to this invention.

In the context of the present invention, the moisture sorption isotherm (or MSI) represents the relationship between the equilibrium water content (wt % water) of the powder and the relative humidity (RH) at which the powder is stored. At a given temperature, by specifying either the RH or the water content of the powder, the other quantity can be readily determined by its MSI. Similarly, for a capsule at a given temperature, by specifying either the RH or the water content of the capsule, the other quantity can be readily determined by its MSI.

As used herein, the term “maximum critical moisture point” is the point at which a dry powder begins to lose its chemical and physical stability (including aerosol properties) and storage stability.

As used herein, the term “minimum critical moisture point” is the point at which a capsule begins to lose its mechanical integrity and/or dispersibility performance of the dry powder is adversely affected. The precise critical moisture (maximum or minimum) point varies from one dry powder formulation to the next and can be readily determined by one skilled in the art, using routine experimentation.

As used herein, the term “critical RH” refers to the level of relative humidity corresponding to a critical moisture point of a particular dry powder. By measuring the moisture sorption isotherm for the powder, one can readily determine: 1) the maximum allowable relative humidity (e.g., the maximum critical RH) sufficient to maintain the powder below its maximum critical moisture point, and 2) the minimum relative humidity (e.g., the minimum critical RH) sufficient to maintain the powder above its minimum critical moisture point.

A “desiccant”, also known as a drying agent, is a material that absorbs or adsorbs water and is used to remove environmental moisture. Desiccants necessarily have a high affinity for water. Examples include calcium oxide, molecular sieves and silica gels. Desiccants described herein primarily act to keep the dry powders sufficiently “dry” (i.e., below the critical moisture point.)

“Mass median diameter” or “MMD” is a measure of particle size, since the powders of the invention are generally polydisperse (i.e., consist of a range of particle sizes). MMD values as reported herein are determined by centrifugal sedimentation, although any number of commonly employed techniques can be used for measuring mean particle size (e.g., electron microscopy, light scattering, laser diffraction).

“Mass median aerodynamic diameter” or “MMAD” is a measure of the aerodynamic size of a dispersed particle. The aerodynamic diameter is used to describe an aerosolized powder in terms of its settling behavior, and is the diameter of a unit density sphere having the same settling velocity, in air, as the particle. The aerodynamic diameter encompasses particle shape, density and physical size of a particle. As used herein, MMAD refers to the midpoint or median of the aerodynamic particle size distribution of an aerosolized powder determined by cascade impaction, unless otherwise indicated. Techniques for measuring MMAD are set forth in the Examples that follow.

### DETAILED DESCRIPTION OF THE INVENTION

According to the invention, a novel procedure for determining, ab initio, the appropriate and optimal capsule filling conditions is set forth herein. Failure to account for the water content of the capsule can expose the powder to significantly higher water contents than originally present, possibly compromising the powder's physical and chemical stability (i.e., wherein the maximum critical moisture point of the powder is exceeded). Capsules filled with dispersible powders according to the invention maintain physical and chemical stability after storage.

Capsules for storing and dispensing pharmaceutical agents are known in the art. Such capsules may carry liquid or solid formulations. For use in the context of the present invention, the capsule must be of a material having moisture sorption characteristics suitable for use with dry powder formulations and mechanical integrity sufficient to withstand a broad range of relative humidities. Desirable capsule characteristics are further discussed in the Examples.

Preferred capsules for use in the present invention are those formed from a watersoluble cellulose derivative, such as those commercially available from Capsugel, a subsidiary of Pfizer, Inc., (NJ, USA) and Shionogi Qualicaps Co., Ltd. (Japan). A preferred process for producing such hard capsules is described in EP 1,044,682 A1, published Oct. 18, 2000. In general, the method of EP '682 comprises the steps of: dispersing a water soluble cellulose derivative in the water; adding and dissolving a gelling agent into the cellulose solution to give a capsule solution; dipping a capsule-forming pin into the capsule solution at a predetermined temperature, then drawing out the pin and inducing gelation of the capsule solution adhering to the pin. This method produces uniform capsules without requiring the strict temperature control associated with prior art manufacturing methods for gelatin capsules. Other materials such as gelatin are suitable for use according to the present invention.

Examples of suitable water-soluble cellulose derivatives include cellulose esters substituted with alkyl groups, especially C₁ to C₄ lower alkyl groups, and/or hydroxyalkyl groups, especially C₁ to C₄ hydroxy lower alkyl groups. Specific examples include hydroxypropyl methyl cellulose (HPMC), hydroxyethyl cellulose, hydroxypropyl cellulose, and hydroxyethyl methyl cellulose. In the context of the present invention, the preferred cellulose derivative is hydroxypropyl methyl cellulose (HPMC).

The capsule material may further include a polymerizing additive or the like. There is no specific limit on the capsule material, so long as it has the requisite chemical and physical characteristics discussed above. Various size capsules are suitable for practice of the present invention, including No. 00, No. 1, No. 2, and No. 3 capsules. HPMC capsules are available in different colors, opacities, and grades, all of which are contemplated for use according to the present invention.

The powder formulations for use with the present invention are known in the art such as those disclosed in WO 96/32149, WO 98/16205, WO 99/16419, WO 01/85136, and WO 01/85137, all of which are hereby incorporated in their entirety by reference. Such formulations may comprise active agents, dispersing agents, and excipients as known in the art. Compositions comprising phospholipids such as those described in WO 99/16419 and WO 01/85136 are particularly preferred. According to preferred embodiments, the dry powder formulation contains a pharmaceutically active agent, including triptans such as sumatriptan, frovatriptan, rizatriptan and zolmatriptan, fluticasone, mometasone, benzodiazepines such as alprazolam and midazolam, nicotine, antibiotics including aminoglycosides, quinolones, macrolides, and beta-lactams such as tobramycin, and ciprofloxacin, anti-infectives such as amphotericin B, dopamine agonists such as L-dopa, proteins and peptides such as LHRH, insulin, and teriparatide.

Once the elements of the formulation are set (i.e., the powder formulation and capsule material selected), the first step is to determine the moisture content of both capsule and powder as a function of RH. At a given temperature, these are given by their respective moisture sorption isotherms (or MSI). As noted above, at a given temperature, the MSI graphically represents the relationship between the equilibrium water content of the powder and the relative humidity (or RH) at which the powder is stored. Thus, by specifying either the RH or the water content of the powder, the other quantity can be readily determined from the MSI.

The respective moisture sorption isotherms are experimentally determined for each element, typically using dynamic vapor sorption (DVS). In addition to measuring the MSI, DVS can be used to estimate the initial RH of the powder and capsule. To do this, the initial mass of the powder (before “drying” at 0% RH in the DVS) is noted. The powder will lose mass during this drying step. After drying is complete, the RH is increased in a stepwise fashion. The RH at which the sample returns to its original mass is the initial RH of the sample. Typically, this value is interpolated from experimentally measured parameters. This estimation is especially useful when it is difficult to estimate the water content from thermogravimeteric analysis (or TGA) data, due to the presence of other volatile compounds, such as blowing agents. The initial water content can then be estimated from the initial RH and the powder's moisture sorption isotherm As discussed above, the relative humidity of a powder is dictated by its water content (and vice-versa). Similarly, the RH of a capsule is dictated by its water content. From their respective MSIs, one can not only estimate the initial water content of both capsule and powder but also mathematically predict the equilibrium RH for a given mass of capsule and mass of powder, which, in turn, can be used to determine the equilibrium moisture content of both materials when placed together. As noted above, it is preferable that at all times, the powder be maintained below its maximum critical moisture point, i.e., that point at which a dry powder begins to lose its chemical and physical stability and storage stability. In some instances, such as with formulations prone to triboelectrification (e.g. formulations comprising sulfate groups), it is also necessary to maintain the powder above its minimum critical moisture point to ensure suitable dispersibilty performance.

Accordingly, from the respective MSIs of capsule and powder, the predicted equilibrium RH and moisture content of capsule and powder can be calculated, preferably using a sorption-desorption moisture transfer model (SDMT) described below. SDMT is not a model per se; it is simply a set of equations based on a mass balance of the total amount of water. It is called a “model” because it uses equations to represent the moisture sorption isotherms of the capsule and powder.

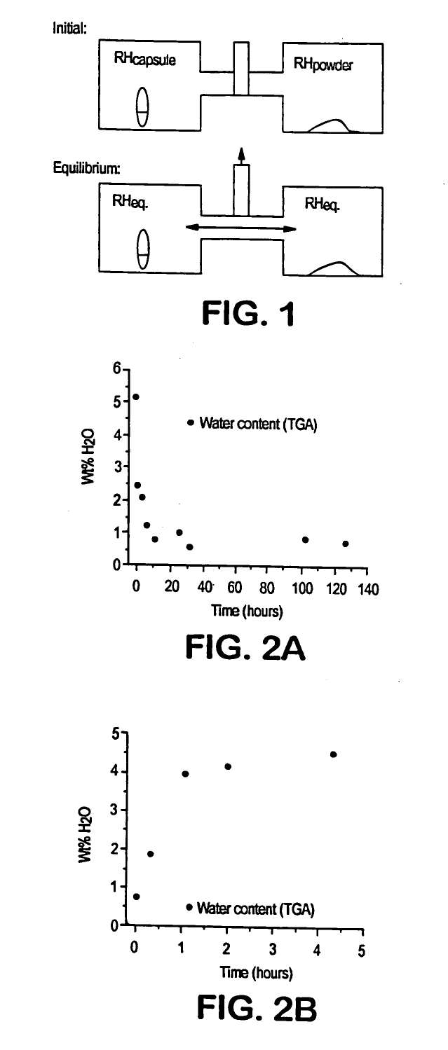

A schematic of the capsule/powder situation is shown in FIG. 1. Initially, the two elements are separately maintained; this separation is represented by two chambers isolated by an impermeable partition. One chamber contains a capsule and the other contains a given mass of powder. The initial moisture contents of each powder and capsule are established by their respective environments; this parameter may be experimentally determined by DVS, as described above. At filling, the capsule and powder are brought together in a common environment; this is represented by the removal of the partition.

Thermodynamic equilibrium requires that the RH, water activity, or chemical potential of water be equal in all phases (i.e., the powder, the capsule, and their relative headspaces). In words, the total mass of water that is initially in the system is given by:

“initial mass of water in capsule+initial mass of water in capsule headspace+initial mass of water in powder+initial mass of water in powder headspace=total mass of water”.

Likewise, the total mass of water that is in the system at equilibrium is given by (i.e., after the partition is removed and sufficient time passes):

“equilibrium mass of water in capsule+equilibrium mass of water in powder+equilibrium mass of water in total headspace=total mass of water”.

Assuming an impermeable container, the total mass of water must be constant; the water is simply redistributed to ensure chemical equilibrium. Thus, the equation becomes:

“initial mass of water in capsule+initial mass of water in capsule headspace+initial mass of water in powder=equilibrium mass of water in capsule+equilibrium mass of water in powder+equilibrium mass of water in total headspace”

The mass of water in a headspace at a given RH and temperature can be easily calculated, according to the following equation, which is based on the ideal gas law:

W_{headspace}(RH)=P^{sat}V/RT×MW_{H2O}×(RH/100),

wherein P^{sat} is the vapor pressure of water at temperature, T, R is the universal gas constant, MW_{H2O} is the molecular weight of water, and V is the volume of the headspace. To come to an equilibrium RH, the RH values of the powder and capsule must both change. Since one material must desorb moisture and the other must sorb moisture, the process and the corresponding mathematical model of the process are known as Sorption-Desorption Moisture Transfer (SDMT).

Likewise, the water contents of the powder and capsule are known as a function of RH, as demonstrated by their respective MSIs. Thus, at any given RH, the total water content in the capsule can be mathematically derived according to the following equation:

W_{capsule}=m_{capsule}(mg dry capsule)×M_{capsule}(mg H₂O/ mg dry capsule),

wherein M_{capsule} is the equilibrium moisture content on a dry basis of the capsule at a given relative humidity.

The total water content in the powder is given by:

W_{powder}=m_{powder}(mg dry capsule)×M_{powder}(mg H₂O/ mg dry capsule),

wherein M_{powder} is the equilibrium moisture content of the powder on a dry basis at a given relative humidity.

MSI can be mathematically represented using several basic functional forms, some of which have a theoretical basis, such as the BET equation, the GAB equation, and the Langmuir equation. (See L. N. Bell et al., “Moisture Sorption”, Amer. Assoc. of Cereal Chemists, 2000, pp. 70-97). In principle, the SDMT can be used with any combination of these equations, though some isotherm equations introduce considerable algebraic complexity into the mathematics.

These equations may be combined to solve for the equilibrium relative humidity, RH_{eq}. This calculated RH_{eq}, in turn, is used to determine the equilibrium moisture content of the powder for a given initial water content of the capsule. Accordingly, based on the critical moisture point of the powder selected, using experimentally measured masses and MSIs of capsule and powder, one can use a SDMT model to pre-determine the optimal initial and equilibrium relative humidity appropriate for a particular powder/capsule combination.

SDMT calculations can be performed for scenarios in which the initial pre-equilibration RH of the capsule is varied. In doing so, a curve can be defined which describes the equilibrium water content of the powder as a function of the initial RH of the capsule.

The RH of the capsule at which the equilibrium water content of the powder is at its maximum critical moisture content is the maximum RH at which the capsules should be pre-equilibrated in order to ensure that the powder water content remains below its critical value (i.e., below the maximum critical moisture point). This is referred to herein as the pre-determined maximum initial capsule RH. It is preferable to select a capsule pre-equilibration RH that is below the maximum value. Since cellulose capsules slowly lose their residual moisture and rapidly take on moisture, pre-equilibration times of at least 48 hours are recommended. Also, mechanical performance of capsules can suffer at low RH.

Over-desiccating the capsules can lead to filling problems, due to static electricity. Static charges may also negatively impact dispersibility of powders. Thus, in addition to a “maximum initial capsule RH”, a minimum initial capsule RH can also be pre-determined. From the maximum and minimum initial RH values, an optimum range of relative humidity conditions for pre-equilibrating the capsules can be determined, ab initio.

With regard to the powders, to minimize moisture content, it is desirable to start with as low an RH as possible. However, in terms of a minimum initial powder RH, a similar phenomenon applies to powders as well as capsules. Over-drying the powders can result in losses in dispersibility and aerosol performance. Accordingly, a suitable minimum initial powder RH can be determined for the powder as well as the capsule. This parameter is referred to herein as the pre-determined ninimum initial powder RH.

From the MSI data, masses of powder and capsule, and SDMT model predictions, the maximum acceptable RH level (i.e., the maximum critical RH) is determined. As noted above, prior to filling, the capsule is pre-equilibrated at an RH level below this critical RH. Similarly, the filling environment is also maintained below this critical RH. In a preferred embodiment, the capsule is filled at the same RH at which it was pre-equilibrated.

Before filling, the dry powder is preferably placed in a container (e.g., a glass vial) that has been stored open in a filling station, typically a Plexiglass box, maintained at the pre-determined RH. Capsules are then filled with the determined mass of powder (typically 1 to 50 mg) in the filling station. The desired fill weight is typically determined by the intended use. However, fill weight can effect the powder's equilibrium moisture content; such effects (if any) may be taken into consideration when determining the fill weight for a particular powder/capsule combination. Capsules are preferably filled individually, i.e., brought one at a time into the filling station, to prevent excessive desiccation of the capsules during filling. Suitable fill weights according to the invention are from 1 mg to 100 mg, preferably 5 mg-75 mg, and most preferably 10 mg 50 mg.

According to a preferred embodiment, the mass ratio of the powder formulation (dry basis): capsule mass (dry) is less than 8.0. More preferably, the mass of powder: capsule mass is less than 2.5, and most preferably this ratio is less than 0.8. Bulk density of the powder is preferably less than 1.0 g/ cm³, preferably less than 0.3 g/ cm³, and most preferably less than 0.1 g/cm³.

To ensure powder stability over long time periods, secondary packaging may be necessary. Secondary packing, such as sealed bottles and foil pouches, with or without desiccants, will have a negligible effect on the initial moisture transfer between powder and capsule. However, such packaging can influence the long-term rate of moisture uptake into the powder and capsule.

Accordingly, in a preferred embodiment, the filled capsule is maintained in a sealed environment to prevent contamination, undue moisture uptake, and the like and to extend shelf-life. A dessicant is included within the sealed environment. Suitable dessicants are known in the art and include, for example, silica gel and indicating silica gel, molecular sieve, and calcium oxide.

A dry powder inhaler (DPI) is a handheld device that delivers a precisely measured dose of active ingredient or medicament into the lungs. The advantage of using a dry powder inhaler is that it is typically breath-activated; thus, one does not have to coordinate activating the inhaler (spraying the medicine) while at the same time inhaling the medication. Instead, one typically breathes in quickly to activate the flow of medication. In this way, the breath-activated discharge of medicine is always coordinated with the inhalation effort.

In a dry powder inhaler the medicament or active ingredient comes in a dry powder form—inside a small capsule, a disk, or a compartment that fits inside the inhaler. As discussed in the background section, many types of dry powder inhalers are described in the art. Of those presently commercially available, each has a different operating method. For example, some have to be loaded each time they are used. Examples of such single-dose DPIs include the Spinhaler® device from Intal (Australia), which coordinates with Spincaps® and utilizes mating screw threads between body elements to advance a propeller, which in turn pierces the capsule to allow medicament to flow into and through the inhalation chamber, Turbospin®, available from PH&T (Italy) which utilizes a telescoping piercing element to access the capsule contents, and the Rotahaler® device (GlaxoSmithKline) which coordinates with Rotocaps® and utilizes a rotational twisting motion to induce the capsule to separate into two halves, thereby releasing the powder medicament therein. Others have disks with a set number of doses (4 or 8), while other DPIs have as many as 200 doses stored in the device. Examples of such multi-dose DPIs include the Turbuhaler® from Astra-Zeneca, the Diskhaler® from Glaxo-Wellcome, and the Clickhaler® from Innovata Biomed. Such devices are disclosed in U.S. Pat. Nos. 4,995,385, 3,991,761, 6,230,707, 6,032,666, 5,873,360, and 4,524,769, hereby incorporated in their entirety by reference.

Despite the difference in specific design and operating mechanism, all DPIs tend to share the following general elements: (1) an actuable device that perforates (e.g., pierces, punctures, tears or otherwise breaks) the seal of the powder container (e.g., the capsule or blister pack) to allow the release of the powder into the device and (2) an inhalation chamber that the powder flows into and through upon application of patient-driven force, such as inspiration pressure, or device-driven force, such as is generated by pressurized gas or vibrating or rotating elements, sufficient to disperse and aerosolize a drug formulation contained within the device. The dry-powder filled capsules of the present invention are intended to coordinate with a multitude of DPIs, regardless of capsule piercing mechanism. Size and shape of the capsule may routinely be adapted to suit a particular device design.

The respirable dry powder formulations of the present invention, when administered pulmonarily, penetrate into the airways of the lungs, enter the circulatory system and achieve effective systemic delivery of the active agent contained within the formulation. Pulmonary administered formulations typically require a much lower dose of active agent those formulations administered orally, primarily due to the loss associated with digestion and degradation for oral dosage forms. The respirable dry powder formulations of the present invention are also suitable for treating local respiratory conditions such as bronchitis, cystic fibrosis, asthma, COPD and the like.

The foregoing description will be more fully understood with reference to the following Examples. Such Examples, are, however, merely representative of preferred methods of practicing the present invention and should not be read as limiting the scope of the invention.

### EXAMPLES

Methods:

Moisture Content Analyses.

The moisture content of the powders is measured by thermogravimetric analysis or experimentally determined from the powder's moisture sorption isotherm, as noted.

Thermogravimetric Analysis (TGA).

The residual solvent content is measured using a TGA-2950 instrument made by TA Instruments. The sample was equilibrated at 30° C. and then heated at a constant rate to a maximum temperature that depended on the sample. The temperature was then held at this temperature for at least 30 minutes. The % weight loss was calculated between the initial and final masses.

Sorption-Desorption Moisture Transfer Model (SDMT).

The equilibrium water content of the dry powders and filled capsules were predicted from the mathematical equations described above.

Dynamic Vapor Sorption (DVS).

The moisture sorption isotherm of each powder at 25° C. was measured using a dynamic vapor sorption (DVS) instrument made by Surface Measurement Systems, UK. This instrument gravimetrically measures uptake and loss of water vapor on a substrate by means of a recording microbalance with a resolution of ±0.1 μg and a daily drift of approximately ±1 μg. In the first step of the experimental run, the sample was dried at 25° C. and 0% RH for at least 600 minutes to bring the sample to near zero wt % H₂O . Then, the instrument was programmed to increase the RH in steps of 5% RH from 0% to 80% RH and decrease the RH in steps of 15% RH from 80% to 0% RH. A criterion of dm/dt=0.005%/min was chosen for the system to hold at each RH step before proceeding to the next RH step. Sample masses between 5 and 20 mg were used in this study.

DVS is also used to estimate the initial relative humidity (RH) of a powder. It is further used to determine the initial moisture content of the powder.

### Example 1

### Capsule Robustness

Experiments to investigate the mechanical integrity of capsules were carried out using size # 2 and #3 HPMC capsules from the suppliers Shionogi (Japan) and Capsugel (NJ, USA), respectively. Capsules were placed in various RH environments, ranging from 0-43% RH for various time periods. In addition, some capsules were placed in secondary packaging and others in environments saturated with a blowing agent, PFOE (perfluorooctyl ethane). The occurrence of shattering and misshapen puncture holes was then assessed by forceful actuation in the TurboSpin® dry powder inhalation device, available from PH&T and the Eclipse® dry powder inhalation device, available from Aventis Pharma (Bridgewater, N.J.).

The results demonstrate that under no conditions tested did the empty HPMC capsules shatter. Furthermore, there were no incidences of abnormal punctures.

Effects of Varying RH on Mechanical Integrity

Following exposure to varying RHs (0-43% at 25° C.) for varying storage times (1 week or 1 month), HPMC capsules were evaluated for brittleness. Brittleness or reduced mechanical integrity can lead to capsule shattering or the formation of a misshapen hole upon puncturing of the capsule, such as occurs upon priming conventional dry powder inhalation devices that utilize capsules as the unit dose package. The result is a possible compromise of aerosol performance and the potential for inhalation of capsule fragments. Thus, brittleness is highly undesirable and conditions that undermine the integrity of the capsules should be avoided.

Varying RH conditions were generated by placing the following saturated salt solutions in vacuum dessicators:

TABLE 1

| Solution | % RH at 20° C. | % RH at 25° C. |
|---|---|---|
| phosphorous pentoxide | 0 | 0 |
| (a strong desiccant) | | |
| lithium bromide | 6.6 | 6.4 |
| lithium chloride | 11.3 | 11.3 |
| lithium iodide | 18.6 | 17.6 |
| potassium acetate | 23.1 | 22.5 |
| potassium fluoride | N/A | 30.8 |
| sodium iodide | 39.7 | 38.2 |
| potassium carbonate | 43.2 | 43.2 |

HPMC capsules were placed therein, the chambers were allowed to come to equilibrium and the final RH % was measured.

Mechanical integrity of the Shionogi #2 capsules was tested with the TurboSpin DPI device using forceful actuation; the Capsugel #3 capsules were tested with the Eclipse DPI device, also using forceful actuation. The procedure called for a rigorous depression of the actuator to cause a high degree of stress on the capsule. Also, a number of capsules were placed in the opposite orientation to that suggested by the device manufacturer so as to introduce a different stress on the capsule. Capsules were then visually inspected for failure.

After one week, Shionogi #2 capsules stored in dessicators were pulled and forcefully actuated with the TurboSpin device. Independent of the storage condition, no capsules shattered. After one month, only the capsules that were stored in the 0% RH environment were tested, again without failure. Shionogi #2 capsules were also subjected to extended storage (one week) either (a) in the presence of PFOE vapor under normal temperature (25° C.) or (b) in the presence of phosphorous pentoxide, a strong desiccant that ensures a 0% RH environment, under extreme temperatures (40° C.). No capsules shattered upon testing.

The Capsugel #3 capsules were similarly tested with the Eclipse DPI, according to the same protocols. Again there was no unsatisfactory tearing, shattering, or brittleness of the capsule; all capsules actuated as expected.

In conclusion, Shionogi #2 HPMC capsules did not shatter under any of the conditions tested. Even at a water content as low as 0.9 wt % water, these capsules did not show any signs of brittleness. These capsules demonstrated reliability at RH environments of less than 1% RH at ambient and elevated temperatures for at least six months. Likewise, Capsugel #3 HPMC capsules did not tear or shatter under any of the conditions tested.

Effects of Secondary Packaging

Several 90 cm³ high density polyethylene (HDPE) bottles filled with 20 Shionogi size #2 HPMC capsules were foil overwrapped with and without dessicant and placed in stability ovens controlled at either 40° C./75% RH or 25° C./60% RH. These capsules were periodically tested over a 6 month period according to the forceful actuation protocols described above. The capsules were shown to maintain their mechanical integrity when stored in secondary packaging for 6 months at 40° C./75% RH and at 25° C./60% RH.

### EXAMPLE 2

### Moisture Transfer Between Capsules and Powders

As noted previously, the present invention provides a novel procedure for determining, ab initio, appropriate and optimal conditions for preparing dry powder filled capsules. The relative humidity of a material is dictated by its water content (and vice-versa). By experimentally measuring respective moisture sorption (or desorption) isotherms using dynamic vapor sorption, one can not only estimate the initial water content of both capsule and powder but also mathematically predict the equilibrium RH of capsule and powder, which, in turn, can be used to determine the equilibrium moisture content of the powder. The calculated equilibrium RH (and corresponding equilibrium moisture point) are used to determine, at the outset, the allowable capsule pre-equilibration RH levels suitable to maintain the powder within its critical moisture points.

Accordingly, the first step in determining the degree of moisture transfer between capsules and powders involves the plotting of the MSI. Next, from the respective MSIs and masses of capsule and powder, the predicted equilibrium RH and moisture content of capsule and powder can be calculated, preferably using the sorption-desorption moisture transfer model (SDMT) described above.

The RH_{eq} calculated according to the SDMT is then used to predict the equilibrium moisture content of the powder. Based on the critical moisture point of the powder selected, using experimentally derived MSI, one can pre-determine the optimum initial and equilibrium relative humidities appropriate for a particular powder/capsule combination.

The following examples describe in detail the determination of the optimum capsule preparation and filling conditions for a particular dry powder formulation.

Determination of Maximum Critical Moisture Point

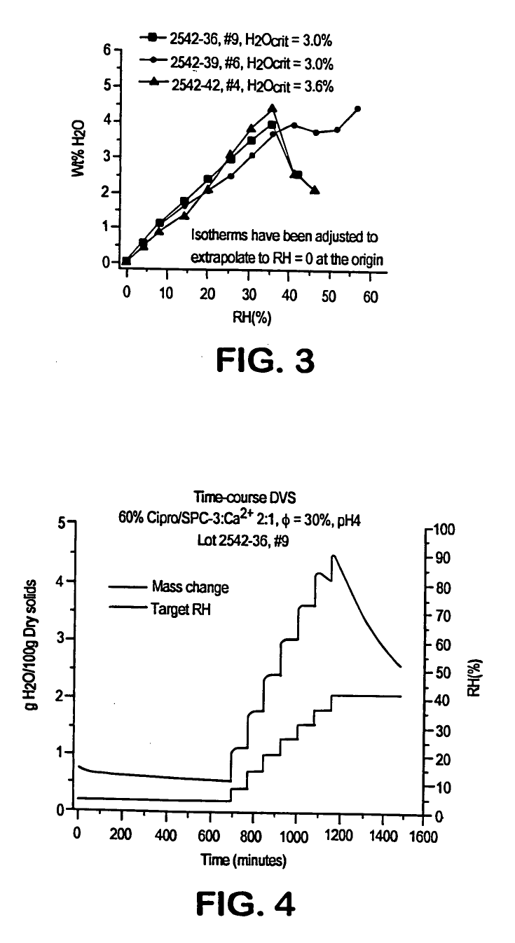

Moisture sorption isotherms for three samples of ciprofloxacin-containing powders made according to the process described in WO 99/16419 were determined by dynamic vapor sorption (DVS), according to the procedures described previously herein. Results are shown in FIG. 3. Each isotherm represents the relationship between the water content of the powder and the RH at which the powder is stored. Thus, by specifying either the RH or the water content of the powder, the other quantity can be readily determined with the MSI. Note, since it is difficult to completely dry these formulations, the lowest RH studied was 5% RH. In order to determine the MSI for these formulations, it was necessary to adjust the isotherms so that the moisture content was 0 wt % H₂O at 0% RH.

In addition to measuring the MSI, DVS was used to estimate the initial RH of the powder. To do this, the initial mass of the powder (before “drying” at 5% RH in the DVS) was noted. The powder loses mass during the drying step. After drying was complete, the RH was increased in a step-wise fashion. The RH at which the sample returned to its original mass was interpolated from the data and deemed the “initial RH” of the sample.

Table 2 below shows the estimated initial RH values for the three samples. This estimation is especially useful when it is difficult to estimate water content from TGA data, due to the presence of other volatile compounds, such as blowing agents. The initial water content can then be estimated from the powder's initial RH and its MSI (FIG. 3).

TABLE 2

| | | Estimated Initial |
|---|---|---|
| | Estimated | Water Content |
| Sample | Initial RH (%) | (wt % H₂O) |
| A | 16.5 | 2.0 |
| B | 15.1 | 1.7 |
| C | 16.6 | 1.6 |

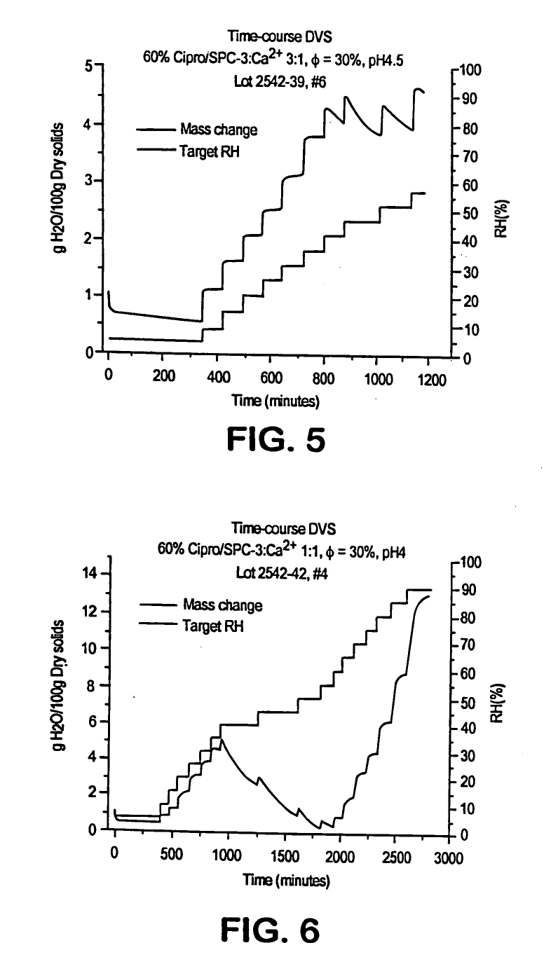

FIGS. 4, 5, and 6 show the time course of moisture sorption for the same three DVS experiments. In contrast to the equilibrium data shown in FIG. 3, these results show the kinetics of moisture uptake during each RH step. At lower RH values, the weight reaches a steady plateau. However, between 30% and 40% RH, the rate of mass sorption becomes negative. It is suspected that the mass loss is induced by crystallization of Ciprofloxacin. In comparison to amorphous materials, crystalline materials generally have a lower capacity for water at a given RH. Thus, crystallization results in the liberation of water. Since crystallization is an undesirable change in the formulation, a critical RH value can be assigned to each of the three sample formulations. In this case, the critical RH is the RH for the step immediately preceding the step in which crystallization began in the DVS. Then, using the MSI of FIG. 3, these critical RH values can be translated into critical moisture criteria (i.e., determining the maximum critical moisture point for the formulation).

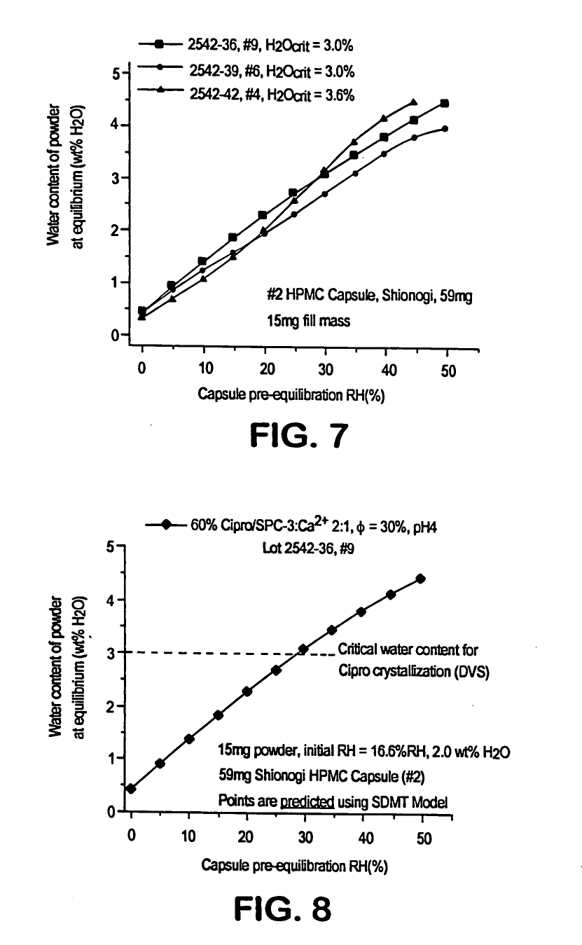

FIG. 7 shows the predictions of an SDMT model. To make the predictions beyond 35% RH, the isotherm of the powder was extrapolated. This model was used to predict the equilibrium water content of the three Ciprofloxacin powders of this example, after filling 15 mg of each powder into Shionogi #2 HPMC capsules that had been preequilibrated at various relative humidities. From this plot, it is apparent that all three powders behave similarly with respect to moisture equilibration with the HPMC capsule. In order to fill all three powders under the same conditions, it is necessary to base the filling decision on the most sensitive powder.

FIG. 8 shows that, for sample A, capsules must be pre-equilibrated and filled below about 30% RH (the maximum critical RH) in order to ensure that the powder water content remains below its maximum critical moisture point (3 wt % H₂O). In order to avoid operating too close to instability, it is recommended that the capsules be pre-equilibrated at no more than 20% RH. Also, though studies herein show that capsule brittleness is not a problem, over-desiccating the capsules may lead to filling problems due to static electricity. Furthermore, over-desiccating the powders can lead to loss in dispersibility and aerosol performance. Accordingly, a minimum threshold RH can be readily determined through mechanical integrity testing as set forth in Example 1 or in aerosol testing as known in the art.

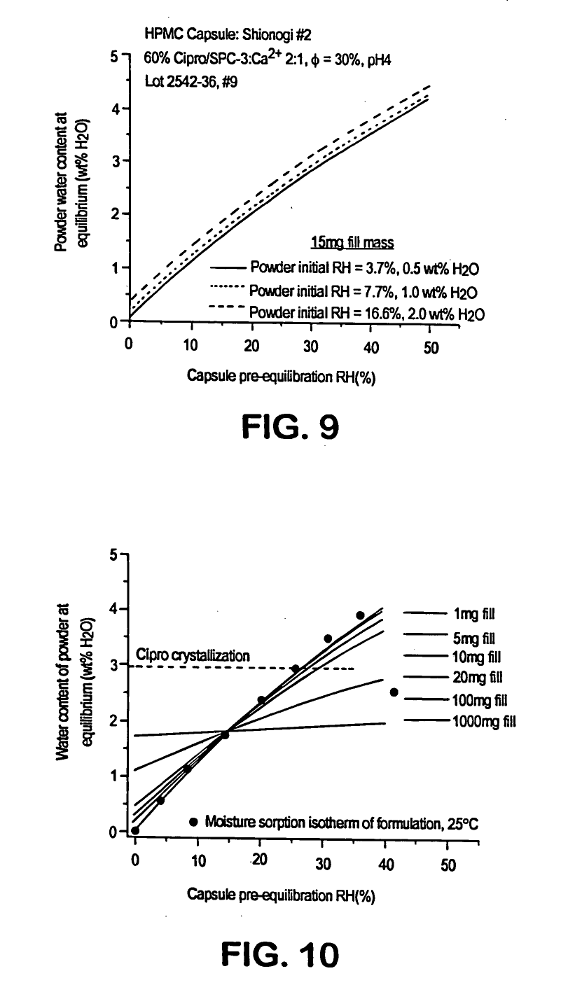

FIG. 9 shows SDMT predictions for capsules filled with the powder of Ciprofloxacin Sample A that has been dried to moisture conditions of 0.5, 1.0 and 2.0 wt % H₂O. As expected, after filling in a capsule that has been pre-equilibrated at a given RH, the powder with the lowest initial water content had the lowest equilibrium water content. However, the equilibrium water content of the powder is only a weak function of the powder's initial water content. That is, the total vertical offset in the curves of FIG. 9 is less than 0.4% wt % H₂O.

FIG. 10 shows the predicted equilibrium water contents of the Ciprofloxacin powder of Sample A, after filling into Shionogi #2 HPMC capsules at fill masses between 1 mg and 1000 mg. Note that all predictions intersect at 15% RH because at this point, the initial RH of the capsule and powder are equal and no moisture transfer occurs. These results illustrate how fill weights affect the powder's equilibrium moisture content. For extremely large fill weights, the water content of the powder is unaffected, as is evident from the nearly horizontal curve of FIG. 10. For practical purposes, moisture is neither transferred to nor from the powder.

For more relevant fill weights (between 1 and 50 mg), the equilibrium moisture content of the powder is dictated by the capsule. For example, Table 3 below shows predictions for filling mg of powder into capsules either at 10% RH or 40% RH. At such a low fill weight, the powder water content approaches the theoretical maximum given by the powder's MSI. In other words, the powder behaves as if it were in an environment at the capsule RH. This is shown graphically in FIG. 10, which has equilibrium moisture sorption data for Sample A. This shows that, if there is insufficient time or data to make model predictions, the worst-case powder water content can be approximated by simply using the powder's MSI.

TABLE 3

| Capsule | Predicted (SDMT) | Theoretical maximum |
|---|---|---|
| pre-equilibration | water content of the | water content of the |
| RH (%) | powder (wt %) | powder (isotherm) (wt %) |
| 10% | 1.25 | 1.27 |
| 40% | 4.06 | 4.20 |

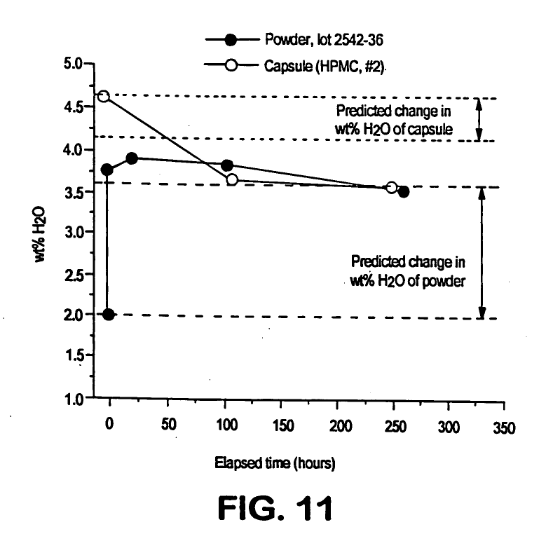

FIG. 11 shows the measured water content of the powder (Ciprofloxacin Sample A) and capsule at various time points after filling; Table 4 shows the numerical results. Table 2 (above) shows the DVS estimated initial water content of the sample to be 2 wt %. Based on this assumption and the average initial residual solvent content measured by TGA, 7.3 wt %, the PFOE content of this sample was estimated to be about 5.3 wt %. Thus, assuming that PFOE content is constant, the residue moisture content can be estimated by subtracting 5.3 wt % from the total loss on drying.

TABLE 4

| | | | wt % water |
|---|---|---|---|
| | Elapsed Time (hrs) | Loss on Drying (%) | (estimated) |
| POWDER | 0.0 | 6.81 | 1.55 |
| | 0.0 | 7.71 | 2.45 |
| | 0.5 | 9.02 | 3.76 |
| | 2.8 | 8.22 | 2.96 |
| | 23.5 | 9.17 | 3.91 |
| | 106.0 | 9.11 | 3.84 |
| | 263.1 | 8.81 | 3.55 |
| CAPSULE | 0.0 | 4.63 | 4.63 |
| | 110.4 | 3.66 | 3.66 |
| | 253.2 | 3.59 | 3.59 |

These results show that, as expected, the powder gains moisture and the capsule loses moisture. Furthermore, the SDMT model predictions agree quite well with measured values. Note that FIG. 11 shows that the capsule and powder approach similar water contents. This is a coincidence since, at equilibrium, the capsule and powder must be at the same RH, but not necessarily the same water content.

The rate of moisture transfer is rapid compared to typical storage time scales. Within an hour after filling, the water content of the powder increases from 2.0 wt % water to 3.8 wt % water. Over time, the powder reaches a maximum water content of 3.9 wt % water, and then begins to decrease slightly. This decrease in water content is likely due to crystallization of Ciprofloxacin over time.

The overall increase in powder water content can be compared to the predictions of the SDMT model using the following pieces of data:

Fill weight=15 mg;

Capsule mass (#2 HPMC, dry)=57.4 mg

Powder initial water content=2.0 wt %;

Powder initial RH=16.6% (determined from powder's MSI);

Capsule initial water content=4.6 wt %;

Capsule initial RH=36.7% (determined from capsule's MSI);

Headspace volume of vial=2.8 ml;

Based on these data, the predicted final RH is 32.6% RH. At this RH, the capsule water content will be 4.2 wt % water and the powder water content will be 3.6 wt % water. These predictions are close to the measured values of 3.6 wt % water and 3.9 wt % water, respectively. FIG. 11 shows that the capsule water content is somewhat lower than expected. This is likely due to sample preparation in a glovebox. When the sample was removed from the capsule for a TGA measurement, the capsule was exposed to <2% RH for 1 to 3 minutes. Likewise, the powder was also desiccated during this short period. Thus, the measured water contents of both the capsule and the powder are likely to be lower than the true values. It is important to note that the final water content of the powder was greater than the value that resulted in Ciprofloxacin crystallization in the DVS experiment.

In sum, the above data demonstrate that:

The initial water content of the capsule (or its pre-equilibration RH) had the greatest impact on the equilibrium water content of the powder; accordingly, the most effective means to modify the equilibrium water content of the powder is to adjust the capsule's pre-equilibration RH.

For typical fill masses, the initial water content of the powder has only modest effect on its equilibrium water content.

For typical fill masses, the relevant fill weights have only minor effect on the equilibrium water content of the powder.

### Example 3

The minimum critical moisture content of the powder is determined through aerosol testing. Capsules are pre-equilibrated at various RH levels and filled with powder formulations. The capsules are then placed in a Turbospin® device and tested for emitted dose. The emitted dose is plotted as a function of powder moisture content. The powder moisture content corresponding to where the emitted dose substantially drops (minimum critical moisture content) is determined from this plot. The powder pre-equilibration RH corresponding to the minimum critical powder moisture content is the minimum equilibrium RH.

The invention has now been described in detail for purposes of clarity and understanding. However, it will be appreciated that certain changes and modifications may be practiced within the scope of the appended claims.

What is claimed:

1. A unit dose package comprising: (a) a dry powder formulation having a maximum critical moisture point and (b) a capsule receiving said dry powder formulation therein and having an initial moisture content such that the moisture content of the powder does not exceed its maximum critical moisture point when the powder is in equilibrium with the capsule, wherein the formulation is storage stable within said capsule at room temperature.

2. The unit dose package of claim 1, wherein the capsule material comprises a cellulose derivative.

3. The unit dose package of claim 2 wherein the cellulose derivative is hydroxypropyl methyl cellulose (HPMC).

4. The unit dose package of claim 1, wherein the dry powder formulation comprises a phospholipid.

5. The unit dose package of claim 4 wherein the dry powder formulation comprises a bulk density of less than 1.0 g/cm³.

6. The unit dose package of claim 4 wherein the dry powder formulation comprises a bulk density of less than 0.3 g/cm³.

7. The unit dose package of claim 4 wherein the dry powder formulation comprises a bulk density of less than 0.1 g/cm³.

8. The unit dose package of claim 4, wherein the dry powder formulation includes a pharmaceutically active agent.

9. The unit dose package of claim 5, wherein the pharmaceutically active agent is selected from the group consisting of sumatriptan, frovatriptan, rizatriptan, zolmatriptan, alprazolam, midazolam, ciprofloxacin, amphotericin B, tobramycin, LHRH, leuprolide, insulin, nicotine and teriparatide.

10. The unit dose package of claim 8 wherein the dry powder formulation further comprises a minimum critical moisture point.

11. The unit dose package of claim 1 wherein the ratio of the mass of the capsule (dry basis) : mass of dry powder formulation is less than 8.0.

12. The unit dose package of claim 1 wherein the ratio of the mass of the capsule (dry basis) : mass of dry powder formulation is less than 2.5.

13. The unit dose package of claim 1 wherein the ratio of the mass of the capsule (dry basis): mass of dry powder formulation is less than 0.8.

14. The unit dose package of claim 8 wherein the package is stored within a sealed environment.

15. The unit dose package of claim 14 wherein a dessicant is stored within the sealed environment.

16. The unit dose package of claim 8 wherein the maximum critical moisture point is less than about 4 wt %.

17. The unit dose package of claim 8 wherein the maximum critical moisture point is less than about 3 wt %.

18. The unit dose package of claim 8 wherein the capsule contains 1 mg-100 mg of said formulation.

19. The unit dose package of claim 8 wherein the capsule contains 5 mg-75 mg of said formulation.

20. The unit dose package of claim 1 wherein the powder is a respirable dry powder formulation.

21. A method of preparing a capsule adapted to contain a dry powder formulation comprising the steps of:
pre-equilibrating the capsule below a maximum relative humidity; and
filling the capsule with the dry powder formulation at a relative humidity pre-selected such that when in equilibrium with the capsule, the moisture content of the powder does not exceed its maximum critical moisture point, thereby ensuring the storage stability of the powder in the capsule.

22. The method of claim 21, wherein the capsule is pre-equilibrated at a relative humidity below 30%.

23. The method of claim 21, wherein the capsule is pre-equilibrated at an RH below 20%.

24. The method of claim 21 wherein the maximum relative humidity is predetermined from the mass and moisture sorption isotherm of the capsule and those of the powder formulation.

25. A dry powder inhaler assembly comprising:
the unit dose package according to claim 1;
an actuable perforating element adapted to access the contents of the capsule of said unit dose package to release the dry powder formulation received therein; and
a mouthpiece in fluid communication with the contents of the capsule through which the released dry powder formulation is inspired into a patient's lungs.

26. The dry powder assembly of claim 25, wherein said perforating element is handactuated.

27. The dry powder assembly of claim 25, wherein said perforating element is actuated by a rotational twisting motion.

28. The dry powder assembly of claim 25, wherein said perforating element is actuated by a horizontal sliding motion.

29. The dry powder assembly of claim 25, wherein said perforating element is actuated by the interconnection of mating screw threads.
